(19) United States
(12) Patent Application Publication
(10) Pub. No.: US 2025/0115537 A1
(43) Pub. Date: Apr. 10, 2025

(54) Compositions Containing Tri-Cyclopentadiene and Processes for Making Same

(71) Applicant: ExxonMobil Chemical Patents Inc., Baytown, TX (US)

(72) Inventors: Terrance C. Osby, Spring, TX (US); Anthony Najem, Houston, TX (US); Nitish Mittal, Houston, TX (US); Kuldeep Wadhwa, Pearland, TX (US); Lucy Gao, Houston, TX (US); Alison M. Miller, Houston, TX (US); Jeffrey M. Sharp, Humble, TX (US); James R. Lattner, La Porte, TX (US)

(21) Appl. No.: 18/730,247

(22) PCT Filed: Dec. 7, 2022
(86) PCT No.: PCT/US2022/081048

§ 371 Date: Jul. 18, 2024

Related U.S. Application Data
(60) Provisional application No. 63/300,806, filed on Jan. 19, 2022.
(60) Provisional application No. 63/316,720, filed on Mar. 4, 2022.

(51) Int. Cl. C07C 13/68

(52) U.S. Cl. CPC C07C 13/68; C07C 2603/66

(57) ABSTRACT

Compositions containing tri-cyclopentadiene and processes for making same. In some embodiments, the composition can include 1 wt % to 6 wt % of TCPD-7; 5 wt % to 25 wt % of TCPD-3; 15 wt % to 30 wt % of TCPD-5; and 55 wt % to 75 wt % of TCPD-1, where all wt % values are based on the combined weight of TCPD-7, TCPD-3, TCPD-5, and TCPD-1 in the composition. In some embodiments, the composition can further include di-cyclopentadiene. In other embodiments, the composition can further include di-cyclopentadiene, tetra-cyclopentadiene, and optionally one or more oligomers heavier than tetra-cyclopentadiene. In some embodiments, the composition can be made via a continuous process.

### CROSS-REFERENCE TO RELATED APPLICATIONS

This application claims priority to and the benefit of U.S. Provisional Application No. 63/300,806 having a filing date of Jan. 19, 2022, and U.S. Provisional Application No. 63/316,720 having a filing date of Mar. 4, 2022, the disclosures of both of which are incorporated herein by reference in their entireties.

### FIELD

Embodiments disclosed herein generally relate to compositions containing tri-cyclopentadiene and processes for making same.

### BACKGROUND

Commercially important cyclic olefin monomer resins typically include readily available and inexpensive cyclic olefins such as di-cyclopentadiene (DCPD). Unfortunately, high purity di-cyclopentadiene melts at 32° C. to 34° C. and is thus a solid at room temperature. The melting point of di-cyclopentadiene can be depressed by adding an adulterant in the form of one or more higher cyclopentadiene oligomers that are copolymerizable with di-cyclopentadiene such as tri-cyclopentadiene (TCPD). As such, commercially available liquid di-cyclopentadiene monomer resins for use in molding of polymer articles typically contain between 10 wt % to 30 wt % of tri-cyclopentadiene and lesser amounts of higher oligomers of cyclopentadiene, e.g., tetra-cyclopentadiene (TeCPD) and penta-cyclopentadiene (PCPD).

The production of tri-cyclopentadiene, however, has been produced via a batch process that is limited to the production of tri-cyclopentadiene on a small scale. Additionally, the batch process is subject to variations in product quality and requires an undesirable amount of plant downtime to accommodate batch switching and equipment cleaning.

There is a need, therefore, for improved processes for making compositions that include tri-cyclopentadiene. Such processes described herein also produce compositions that include a unique mixture of tri-cyclopentadiene isomers.

### SUMMARY

Compositions containing tri-cyclopentadiene isomers and processes for making same are provided. In some embodiments, the composition can include:
0.1 wt % to 6 wt % of TCPD-7 having a structure of

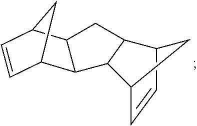 [SMILES: C1=CC2CC1C1CC3C4C=CC(C4)C3C21]

5 wt % to 25 wt % of TCPD-3 having a structure of

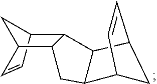 [SMILES: C1=CC2CC1C1CC3C4C=CC(C4)C3C21]

15 wt % to 30 wt % of TCPD-5 having a structure of

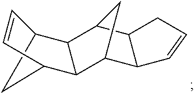 [SMILES: C1=CC2C(C1)C1CC2C2C3C=CC(CC3)C12]

and
55 wt % to 75 wt % of TCPD-1 having a structure of

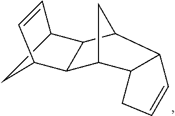 [SMILES: C1=CC2C(C1)C1CC2C2C3C=CC(CC3)C12]

where all wt % values are based on the combined weight of TCPD-7, TCPD-3, TCPD-5, and TCPD-1 in the composition.

In some embodiments, a process for making tri-cyclopentadiene can include (I) feeding a hydrocarbon feed comprising cyclopentadiene and di-cyclopentadiene into a reaction zone; (II) subjecting the hydrocarbon feed to reaction conditions sufficient to effect reaction between the cyclopentadiene and the di-cyclopentadiene within the reaction zone to produce an effluent that can include residual cyclopentadiene, residual di-cyclopentadiene, tri-cyclopentadiene, and tetra-cyclopentadiene; (III) separating from the effluent a recycle stream that can include residual cyclopentadiene and residual di-cyclopentadiene and a first product stream that can include residual di-cyclopentadiene, tri-cyclopentadiene, and tetra-cyclopentadiene; and (IV) feeding at least a portion of the recycle stream into the reaction zone.

In other embodiments, a process for making tri-cyclopentadiene can include (I) feeding a hydrocarbon feed comprising cyclopentadiene and di-cyclopentadiene into a reaction zone; (II) subjecting the hydrocarbon feed to reaction conditions sufficient to effect reaction between the cyclopentadiene and the di-cyclopentadiene within the reaction zone to produce an effluent that can include residual cyclopentadiene, residual di-cyclopentadiene, tri-cyclopentadiene, and tetra-cyclopentadiene; (III) separating from the effluent a recycle stream that can include residual cyclopentadiene and a first portion of the residual di-cyclopentadiene, a second product that can include a second portion of the residual di-cyclopentadiene and can be rich in tri-cyclopentadiene and lean in tetra-cyclopentadiene, and a third product that can be rich in tetra-cyclopentadiene and lean in tri-cyclopentadiene; and (IV) feeding at least a portion of the recycle stream into the reaction zone.

### BRIEF DESCRIPTION OF THE DRAWINGS

So that the manner in which the above recited features of the present invention can be understood in detail, a more particular description of the invention, briefly summarized above, may be had by reference to embodiments, some of which are illustrated in the appended drawings. It is to be noted, however, that the appended drawings illustrate only typical embodiments of this invention and are therefore not to be considered limiting of its scope, for the invention may admit to other equally effective embodiments.

FIG. 1 depicts an illustrative system for making a composition containing tri-cyclopentadiene that includes an optional mixing zone, a reaction zone, and a separation zone, according to one or more embodiments of this disclosure.

FIG. 2 depicts an illustrative system for making a composition containing tri-cyclopentadiene that includes the optional mixing zone, the reaction zone, and the separation zone shown in the system depicted in FIG. 1 and further includes one or more optional oxygen separation zones, dimerization zones, pre-heat zones, and/or separation zones and/or one or more additional antioxidant feeds and/or diluent feeds, according to one or more embodiments of this disclosure.

FIG. 3 depicts an illustrative system for making a composition containing tri-cyclopentadiene that includes the optional mixing zone and the reaction zone shown in the system depicted in FIG. 1 and a separation zone that includes a divided wall distillation column, according to one or more embodiments of this disclosure.

### DETAILED DESCRIPTION

It is to be understood that the following disclosure describes several exemplary embodiments for implementing different features, structures, and/or functions of the invention. Exemplary embodiments of components, arrangements, and configurations are described below to simplify the present disclosure; however, these exemplary embodiments are provided merely as examples and are not intended to limit the scope of the invention. Additionally, the present disclosure may repeat reference numerals and/or letters in the various exemplary embodiments and across the Figures provided herein. This repetition is for the purpose of simplicity and clarity and does not in itself dictate a relationship between the various exemplary embodiments and/or configurations discussed in the Figures. Moreover, the exemplary embodiments presented below can be combined in any combination of ways, i.e., any element from one exemplary embodiment can be used in any other exemplary embodiment, without departing from the scope of the disclosure.

The indefinite article “a” or “an”, as used herein, means “at least one” unless specified to the contrary or the context clearly indicates otherwise. Thus, embodiments using “a separator” include embodiments where one or two or more separators are used, unless specified to the contrary or the context clearly indicates that only one separator is used. Likewise, embodiments using “a separation stage” include embodiments where one or two or more separation stages are used, unless specified to the contrary.

Certain embodiments and features have been described using a set of numerical upper limits and a set of numerical lower limits. It should be appreciated that ranges including the combination of any two values, e.g., the combination of any lower value with any upper value, the combination of any two lower values, and/or the combination of any two upper values are contemplated unless otherwise indicated. Certain lower limits, upper limits and ranges appear in one or more claims below. All numerical values are “about” or “approximately” the indicated value, and take into account experimental error and variations that would be expected by a person having ordinary skill in the art.

As used herein, the term “hydrocarbon” means a class of compounds containing hydrogen bound to carbon. The term “Cₙ” hydrocarbon means hydrocarbon having n carbon atom(s) per molecule, where n is a positive integer. The term “Cₙ₊” hydrocarbon means hydrocarbon having at least n carbon atom(s) per molecule, where n is a positive integer. The term “Cₙ” hydrocarbon means hydrocarbon having no more than n number of carbon atom(s) per molecule, where n is a positive integer. “Hydrocarbon” encompasses (i) saturated hydrocarbon, (ii) unsaturated hydrocarbon, and (iii) mixtures of hydrocarbons, including mixtures of hydrocarbon compounds (saturated and/or unsaturated), including mixtures of hydrocarbon compounds having different values of n.

In this disclosure, any material or compound illustrated by a chemical structure shall include, in addition to the chemical structure, any and all optical isomer(s) thereof, unless clearly specified otherwise. Thus, each of the exo-DCPD, endo-DCPD, TCPD-1, TCPD-2, TCPD-3, TCPD-4, TCPD-5, TCPD-6, TCPD-7, TCPD-8, and the like, as defined and/or described in this disclosure shall include, in addition to the illustrated structure therefor, any and all optical isomer(s) of the illustrated structure.

In this disclosure, “cyclopentadiene” or “CPD” interchangeably means cyclopenta-1,3-diene,

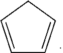

In this disclosure, “di-cyclopentadiene”, or “dicyclopentadiene”, or “DCPD” interchangeably means a molecule or a mixture of molecules each having a chemical formula C₁₀H₁₂ and obtainable via a Diels-Alder reaction between two CPD molecules. A DCPD molecule in this disclosure can be or can include an exo-DCPD stereo isomer, an endo-DCPD stereo isomer, or a mixture of both at any proportion. Exo-DCPD and endo-DCPD isomers can have structures as illustrated below, respectively:

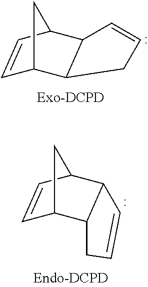 [SMILES: C1=CC2C3C=CC(CC3)C2C1 ; C1=CC2C3C=CC(CC3)C2C1]

In this disclosure, “tri-cyclopentadiene”, or “tricyclopentadiene”, or “TCPD” interchangeably means a molecule or a mixture of molecules each having a chemical formula C₁₅H₁₈ and obtainable via a Diels-Alder reaction between a DCPD molecule and a CPD molecule. Thus, a TCPD in this disclosure can be or can include a single TCPD isomer, or a mixture of any two or more TCPD isomers. TCPD molecules can include the 6,5,6-isomers

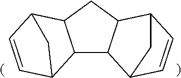 [SMILES: C1=CC2CC1C1CC3C4C=CC(C4)C3C21]

and the 6,6,5-isomers

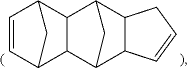 [SMILES: C1=CC2C(C1)C1CC2C2C3C=CC(C3)C12]

each of which can include multiple stereo isomers. A TCPD can be or can include one or more of the following isomers TCPD-1, TCPD-2, TCPD-3, TCPD-4, TCPD-5, TCPD-6, TCPD-7, and TCPD-8, at various quantities thereof, which can be obtained via Diels-Alder reactions between a CPD molecule and an identified DCPD isomer at the identified DCPD reaction bond, shown in Table I below. A composition containing TCPD in this disclosure can include a single TCPD isomer, but typically include a mixture of multiple TCPD isomers.

TABLE I

| TCPD Isomer | | | Diels-Alder Reaction of CPD and | |
|---|---|---|---|---|
| Name | Structure | Type | DCPD Isomer | DCPD Reaction Bond |
| TCPD-1 | 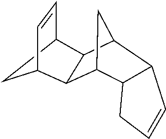 [SMILES: C1=CC2C(C1)C1CC2C2C3C=CC(CC3)C12] | (6,6,5) | endo-DCPD | norbornyl-adduct |
| TCPD-2 | 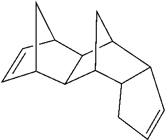 [SMILES: C1=CC2C(C1)C1CC2C2C3C=CC(CC3)C12] | (6,6,5) | endo-DCPD | norbornyl-adduct |
| TCPD-3 | 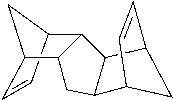 [SMILES: C1=CC2CC1C1CC3C4C=CC(C4)C3C21] | (6,5,6) | endo-DCPD | cyclopentyl-adduct |
| TCPD-4 | 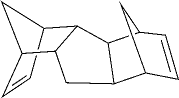 [SMILES: C1=CC2CC1C1CC3C4C=CC(C4)C3C21] | (6,5,6) | endo-DCPD | cyclopentyl-adduct |
| TCPD-5 | 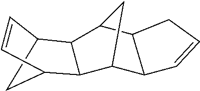 [SMILES: C1=CC2C(C1)C1CC2C2C3C=CC(C3)C12] | (6,6,5) | exo-DCPD | norbornyl-adduct |
| TCPD-6 | 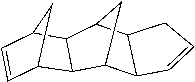 [SMILES: C1=CC2C(C1)C1CC2C2C3C=CC(C3)C12] | (6,6,5) | exo-DCPD | norbornyl-adduct |
| TCPD-7 | 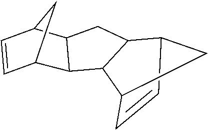 [SMILES: C1=CC2CC1C1CC3C4C=CC(C4)C3C21] | (6,5,6) | exo-DCPD | cyclopentyl-adduct |
| TCPD-8 | 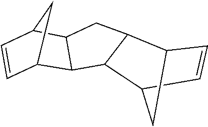 [SMILES: C1=CC2CC1C1CC3C4C=CC(C4)C3C21] | (6,5,6) | exo-DCPD | cyclopentyl-adduct |

In this disclosure, “tetra-cyclopentadiene”, or “tetracyclopentadiene”, or “TeCPD” interchangeably means a molecule or a mixture of molecules each having a chemical formula C₂₀H₂₄ and obtainable via a Diels-Alder reaction between a TCPD molecule and a CPD molecule. Thus, a TeCPD in this disclosure can be a single TeCPD isomer, or a mixture of any two or more TeCPD isomers.

The term “rich” when used in phrases such as “X-rich” or “rich in X” means, with respect to an outgoing product obtained from a device, e.g., a separation zone, that the product comprises material X at a concentration higher than in the feed material fed to the same device from which the product is derived. The term “lean” when used in phrases such as “X-lean” or “lean in X” means, with respect to an outgoing product obtained from a device, e.g., a separation zone, that the product comprises material X at a concentration lower than in the feed material fed to the same device from which the product is derived.

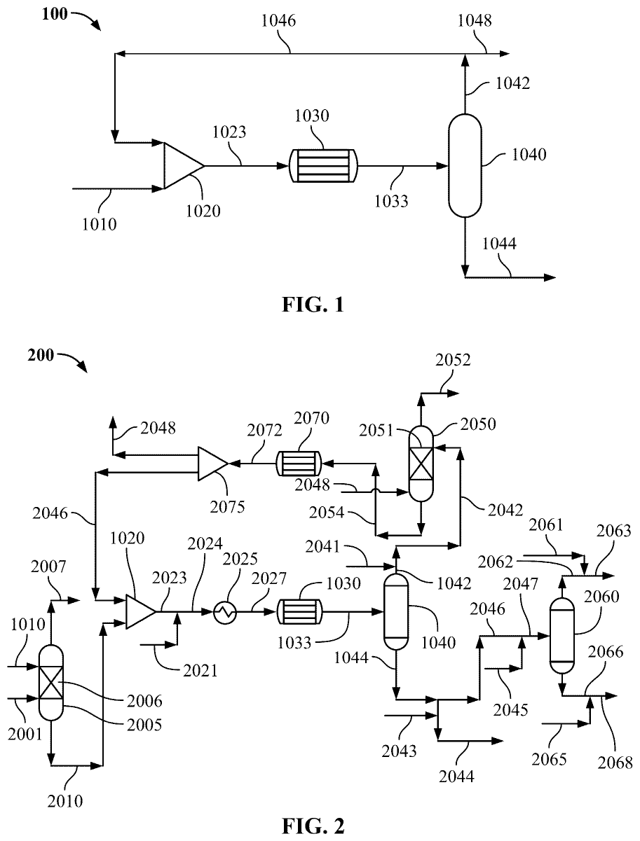

FIG. 1 depicts an illustrative system 100 for making a composition containing tri-cyclopentadiene that includes an optional mixing zone 1020, a reaction zone 1030, and a separation zone 1040, according to one or more embodiments. In some embodiments, a hydrocarbon feed via line 1010 and a recycle stream via line 1046 can be introduced into the optional mixing zone 1020 to produce a combined or mixed feed via line 1023 and the combined or mixed feed via line 1023 can be introduced into the reaction zone 1030. In other embodiments, the hydrocarbon feed via line 1010 and the recycle stream via line 1046 can be separately introduced into the reaction zone 1030. The hydrocarbon feed in line 1010 and/or the recycle stream in line 1046 can be or can include, but is not limited to, di-cyclopentadiene or a mixture of di-cyclopentadiene and cyclopentadiene.

In some embodiments, the combination of the hydrocarbon feed in line 1010 and the recycle stream in line 1046 fed into the reaction zone 1030, whether as a mixture or separately, can include 0.1 wt %, 1 wt %, 3 wt %, or 5 wt % to 10 wt %, 30 wt %, or 50 wt % of the cyclopentadiene and 50 wt %, 70 wt %, or 90 wt % to 95 wt %, 97 wt %, 99 wt %, 99.9 wt % or more of the di-cyclopentadiene, based on the total combined weight of the cyclopentadiene and the di-cyclopentadiene in the hydrocarbon feed and the recycle stream. In other embodiments, the combination of the hydrocarbon feed in line 1010 and the recycle stream in line 1046 fed into the reaction zone 1030, whether as a mixture or separately, can include 0.1 wt %, 1 wt %, 3 wt %, or 5 wt % to 10 wt %, 30 wt %, or 50 wt % of the cyclopentadiene and 50 wt %, 70 wt %, or 90 wt % to 95 wt %, 97 wt %, 99 wt %, 99.9 wt % or more of the di-cyclopentadiene, based on the total combined weight of the hydrocarbon feed and the recycle stream. Di-cyclopentadiene in the hydrocarbon feed in line 1010 and the recycle stream in line 1046 can be or can include endo-di-cyclopentadiene, exo-di-cyclopentadiene, or a mixture thereof. In some embodiments, di-cyclopentadiene in the hydrocarbon feed in line 1010 and the recycle stream in line 1046 can include a mixture of endo-di-cyclopentadiene and exo-di-cyclopentadiene. In such embodiment, a weight ratio of the endo-di-cyclopentadiene to the exo-di-cyclopentadiene introduced into the reaction zone 1030 can be in a range of from 4.5, 5, 5.5, 6, or 7 to 8, 9, 9.5, 10, 12, 14, 16, 18, or 20.

In some embodiments, the hydrocarbon feed in line 1010 and the recycle stream in line 1046 fed into the reaction zone 1030, whether as a mixture or separately, can include ≥90 wt %, ≥93 wt %, ≥95 wt %, ≥97 wt %, ≥98 wt %, or ≥99 wt % of di-cyclopentadiene and ≤10 wt %, ≤7 wt %, ≤5 wt %, ≤3 wt %, ≤2 wt %, or ≤1 wt % of cyclopentadiene based on the total combined amount of the cyclopentadiene and the di-cyclopentadiene in the hydrocarbon feed and the recycle stream. In other embodiments, the hydrocarbon feed in line 1010 and the recycle stream in line 1046 fed into the reaction zone 1030, whether as a mixture or separately, can include ≥90 wt %, ≥93 wt %, ≥95 wt %, ≥97 wt %, ≥98 wt %, or ≥99 wt % of di-cyclopentadiene and ≤10 wt %, ≤7 wt %, ≤5 wt %, ≤3 wt %, ≤2 wt %, or ≤1 wt % of cyclopentadiene based on the total combined weight of the hydrocarbon feed and the recycle stream. In some embodiments, a molar ratio of di-cyclopentadiene to cyclopentadiene fed into the reaction zone 1030 can be in a range of from 0.5, 1, 5, 10, 25, 50, 75, or 100 to 150, 200, 250, 300, 350, 400, 450, or 499.5. In other embodiments, a molar ratio of di-cyclopentadiene to cyclopentadiene fed into the reaction zone 1030 can be in a range of from 4.5, 5, 7, 9, 11, 13, 15, 17, or 20 to 23, 27, 29, 33, 37, 41, 45, 47, 48, 49, or 49.5.

In some embodiments, the hydrocarbon feed in line 1010 and/or the recycle stream in line 1046 fed into the reaction zone 1030 can include, in addition to cyclopentadiene and di-cyclopentadiene, tri-cyclopentadiene (preferably at a relatively low concentration, e.g., from 0.01 wt %, 0.05 wt %, 0.1 wt %, 0.5 wt %, or 1 wt % to 1.5 wt %, 2 wt %, 2.5 wt %, 3 wt %, 4 wt %, or 5 wt %, based on the combined weight of the hydrocarbon feed and the recycle stream). In some embodiments, if the hydrocarbon feed in line 1010 and/or the recycle stream in line 1046 fed into the reaction zone 1030 include, in addition to cyclopentadiene and di-cyclopentadiene, tri-cyclopentadiene, the amount of tri-cyclopentadiene can be ≤5 wt %, ≤4 wt %, ≤3 wt %, ≤2 wt %, ≤1 wt %, or ≤0.5 wt %, based on the combined weight of the hydrocarbon feed and the recycle stream. The presence of tri-cyclopentadiene can be particularly likely if the hydrocarbon feed in line 1010 and/or the recycle stream in line 1046 passes through an optional preheat zone where it can be preheated to a temperature in the vicinity of the temperature within the reaction zone 1030.

The hydrocarbon feed in line 1010 and the recycle stream in line 1046 or the mixture thereof in line 1023 can be heated within the reaction zone 1030 to a temperature in a range of from 155° C., 160° C., 165° C., or 170° C. to 180° C., 185° C., 190° C., or 195° C. In some embodiments, the hydrocarbon feed in line 1010 and the recycle stream in line 1046 or the mixture thereof in line 1023 can be heated within the reaction zone 1030 by heat generated from the reactions occurring therein. In some embodiments, the hydrocarbon feed in line 1010 and/or the recycle stream in line 1046 or the mixture thereof can be at a temperature in a range of from 145° C., 150° C., 155° C., or 160° C. to 165° C., 170° C., 175° C., or 180° C. when introduced into the reaction zone 1030. In some embodiments, the hydrocarbon feed in line 1010 and/or the recycle stream in line 1046, or the mixture thereof in line 1023 can be preheated in an optional preheat zone, if needed, to heat the feed(s) to a desired temperature for introduction into the reaction zone 1030. In other embodiments, the hydrocarbon feed in line 1010 can be obtained from an integrated upstream process that produces a suitable hydrocarbon feed having a sufficient elevated temperature such that the preheating step can be omitted.

The mixture of the hydrocarbon feed and the recycle stream can be subjected to reaction conditions within the reaction zone 1030 sufficient to effect reaction between the cyclopentadiene and the di-cyclopentadiene to produce an effluent that can include tri-cyclopentadiene (e.g., a mixture of two or more tri-cyclopentadiene isomers), tetra-cyclopentadiene, optionally one or more oligomers heavier than tetra-cyclopentadiene, e.g., penta-cyclopentadiene, residual cyclopentadiene, residual di-cyclopentadiene, or a mixture thereof. The effluent can be recovered via line 1033 from the reaction zone 1030.

When the hydrocarbon feed in line 1010 and the recycle stream in line 1046 provide a mixed feed in line 1023 or upon separate introduction into the reaction zone 1030 provide a mixture having a concentration of ≥97 wt %, ≥98 wt %, or ≥99 wt % of di-cyclopentadiene based on the combined amount of di-cyclopentadiene and any cyclopentadiene a portion of the di-cyclopentadiene can back crack via a retro Diels-Alder reaction to produce cyclopentadiene, thereby increasing the concentration of cyclopentadiene within the reaction zone 1030 available for reaction with the di-cyclopentadiene. For example, when the hydrocarbon feed in line 1010 and the recycle stream in line 1046 provide a mixed feed in line 1023 or upon separate introduction into the reaction zone 1030 provide a mixture that includes ≥97.5 wt %, ≥98 wt %, or ≥99 wt % of di-cyclopentadiene based on the combined amount of di-cyclopentadiene and any cyclopentadiene and the reaction zone 1030 is at a temperature of 175° C. an equilibrium amount of cyclopentadiene in a range of from 3 wt % to 4 wt %, e.g., 3.5 wt %, can be obtained.

The reaction zone 1030 can be operated at a sufficient pressure to maintain a liquid phase of the contents therein. In some embodiments, the reaction zone 1030 can be operated at a pressure of >138 kPa-absolute (kPa-a), >206 kPa-a, or >653 kPa-a and up to any pressure desired, e.g., up to a pressure of 1,540 kPa-a or 4,485 kPa-a, when operated at a temperature of 160° C. In some embodiments, the reaction zone 1030 can be operated at a pressure >255 kPa-absolute (kPa-a), >351 kPa-a, or >653 kPa-a and up to any pressure desired, e.g., up to a pressure of 1,420 kPa-a or 4,400 kPa-a, when operated at a temperature of 190° C.

In some embodiments, the reaction zone 1030 can be an isothermal reaction zone, an adiabatic reaction zone, or a combination thereof. For example, in some embodiments, the reaction zone 1030 can include one or more isothermal zones and one or more adiabatic zones serially arranged with respect to one another. In some embodiments, the reaction zone can include one or more internal structures configured to provide a desired hydrodynamic behavior of the reactants flowing therethrough. In other embodiments, the reaction zone can be free of any internal structure configured to provide a desired hydrodynamic behavior of the reactants flowing therethrough.

The reaction zone 1030 can be sized and the flowrate of the feed therethrough can be controlled to provide a relatively low conversion rate of di-cyclopentadiene per pass through the reaction zone 1030. In some embodiments, the residence time of the hydrocarbon feed and the recycle stream, whether introduced as a mixture or separately into the reaction zone 1030, can be in a range of from 10 minutes, 15 minutes, 20 minutes, 25 minutes, 30 minutes, or 35 minutes to 40 minutes, 45 minutes, 50 minutes, 60 minutes 65 minutes, or 70 minutes.

By maintaining a relatively low conversion rate of di-cyclopentadiene within the reaction zone 1030 the amount of higher oligomers of cyclopentadiene, e.g., tetra-cyclopentadiene and penta-cyclopentadiene, can be reduced or minimized. In some embodiments, the amount of di-cyclopentadiene within the reaction zone 1030 converted to tri-cyclopentadiene and tetra-cyclopentadiene, and optionally one or more oligomers heavier than tetra-cyclopentadiene can be <15 wt %, <12 wt %, <10 wt %, <9 wt %, <8 wt %, or <7.5 wt % based on the weight of di-cyclopentadiene introduced into the reaction zone 1030.

Tri-cyclopentadiene in the effluent in line 1033 can be or can include, but is not limited to, TCPD-1, TCPD-2, TCPD-3, TCPD-4, TCPD-5, TCPD-6, TCPD-7, TCPD-8, or any mixture thereof. In some embodiments, the tri-cyclopentadiene in the effluent in line 1033 can include, but is not limited to, a mixture of TCPD-7, TCPD-3, TCPD-5, and TCPD-1. In other embodiments, the tri-cyclopentadiene in the effluent in line 1033 can include, but is not limited to, a mixture of TCPD-7, TCPD-3, TCPD-5, TCPD-1, TCPD-8, and TCPD-6.

In some embodiments, the effluent in line 1033 can include 0.1 wt %, 1 wt %, 1.5 wt %, 2 wt %, or 3 wt % to 4 wt %, 4.5 wt %, 5 wt %, 5.5 wt %, or 6 wt % of TCPD-7, based on the combined weight of the TCPD-7, TCPD-3, TCPD-5, and TCPD-1. In some embodiments, the effluent in line 1033 can include 5 wt %, 6 wt %, 7 wt %, 8 wt %, or 9 wt % to 10 wt %, 13 wt %, 15 wt %, 20 wt %, or 25 wt % of TCPD-3, based on the combined weight of TCPD-7, TCPD-3, TCPD-5, and TCPD-1. In some embodiments, the effluent in line 1033 can include 10 wt %, 12 wt %, 15 wt %, 17 wt %, 19 wt %, or 21 wt % to 23 wt %, 25 wt %, 27 wt %, or 30 wt % of TCPD-5, based on the combined weight of TCPD-7, TCPD-3, TCPD-5, and TCPD-1. In some embodiments, the effluent in line 1033 can include 55 wt %, 57 wt %, 60 wt %, 62 wt %, or 64 wt % to 66 wt %, 68 wt %, 70 wt %, 72 wt %, or 75 wt % of TCPD-1, based on the combined weight of TCPD-7, TCPD-3, TCPD-5, and TCPD-1. In some embodiments, the effluent in line 1033 can be free of or can include ≤1 wt %, ≤0.5 wt %, or ≤0.1 wt % of TCPD-8, based on the combined weight of TCPD-7, TCPD-3, TCPD-5, and TCPD-1. In some embodiments, the effluent in line 1033 can be free of or can include ≤1 wt %, ≤0.5 wt %, or ≤0.1 wt % of TCPD-6, based on the combined weight of TCPD-7, TCPD-3, TCPD-5, and TCPD-1. In some embodiments, the effluent in line 1033 can be free of or can include ≤1 wt %, ≤0.5 wt %, or ≤0.1 wt % of TCPD-2, based on the combined weight of TCPD-7, TCPD-3, TCPD-5, and TCPD-1. In some embodiments, the effluent in line 1033 can be free of or can include ≤1 wt %, ≤0.5 wt %, or ≤0.1 wt % of TCPD-4, based on the combined weight of TCPD-7, TCPD-3, TCPD-5, and TCPD-1.

In some embodiments, the effluent in line 1033 can include ≥10 wt %, ≥12 wt %, ≥14 wt %, ≥16 wt %, ≥18 wt %, or ≥20 wt % of TCPD-5, based on the combined weight of TCPD-7, TCPD-3, TCPD-5, and TCPD-1. In some embodiments, the effluent in line 1033 can include ≤75 wt %, ≤73 wt %, ≤70 wt %, ≤68 wt %, ≤67 wt %, ≤66 wt %, or ≤65 wt % of TCPD-1, based on the combined weight of TCPD-7, TCPD-3, TCPD-5, and TCPD-1. In some embodiments, the effluent in line 1033 can include 0.1 wt %, 1 wt %, 1.5 wt %, 2 wt %, or 3 wt % to 4 wt %, 4.5 wt %, 5 wt %, 5.5 wt %, or 6 wt % of TCPD-7, 5 wt %, 6 wt %, 7 wt %, 8 wt %, or 9 wt % to 10 wt %, 13 wt %, 15 wt %, 20 wt %, or 25 wt % of TCPD-3, ≥10 wt %, ≥12 wt %, ≥14 wt %, ≥16 wt %, ≥18 wt %, or ≥20 wt % of TCPD-5, ≤75 wt %, ≤73 wt %, ≤70 wt %, ≤68 wt %, ≤67 wt %, ≤66 wt %, or ≤65 wt % of TCPD-1, can be free of or can include ≤1 wt %, ≤0.5 wt %, or ≤0.1 wt % of TCPD-8, can be free of or can include ≤1 wt %, ≤0.5 wt %, or ≤0.1 wt % of TCPD-6, can be free of or can include ≤1 wt %, ≤0.5 wt %, or ≤0.1 wt % of TCPD-2, and can be free of or can include ≤1 wt %, ≤0.5 wt %, or ≤0.1 wt % of TCPD-4, where all weight percent values are based on the combined weight of TCPD-7, TCPD-3, TCPD-5, and TCPD-1. In some embodiments, the TCPD-7, TCPD-3, TCPD-5, and TCPD-1 can constitute at least 95 wt %, at least 96 wt %, at least 97 wt %, at least 98 wt %, at least 99 wt %, or at least 99.5 wt % of all tri-cyclopentadiene in the effluent.

The effluent in line 1033 can further include residual cyclopentadiene, residual di-cyclopentadiene, tetra-cyclopentadiene, one or more oligomers heavier than tetra-cyclopentadiene, one or more light compounds present in the hydrocarbon feed 1010 and/or the recycle feed 1046 and/or produced within the reaction zone 1030, or a mixture thereof. The one or more light compounds that can be present in the hydrocarbon feed 1010 and/or the recycle feed 1046 and/or can be produced within the reaction zone 1030 can be or can include, but are not limited to, isoprene, piperylene, benzene, butadiene, pentene, or any mixture thereof.

In some embodiments, the effluent in line 1033 can be introduced into the separation zone 1040. As shown, an overhead or recycle stream via line 1042 and a bottoms or first product stream via line 1044 can be recovered from the separation zone 1040. The separation zone 1040 can be or can include any suitable separation technique. In some embodiments, suitable separation techniques can be or can include, but are not limited to, distillation, evaporation, crystallization, or a combination thereof. In some embodiments, the separation zone 1040 can be or can include one or more distillation columns, one or more divided wall distillation columns, or a combination thereof.

In some embodiments, the separation zone 1040 can include one or more internal structures to facilitate separation of the overhead and the bottoms from the effluent in line 1033. The internal structure(s) can facilitate vapor/liquid separation and/or liquid collection. Illustrative internal structures can be or can include, but are not limited to, trays, grids, packing, or any combination thereof. Illustrative trays can include, but are not limited to, fixed valve trays, jet tab trays, sieve trays, dual flow trays, baffle trays, angle iron trays, draw off trays, shed deck trays, disk trays, donut trays, side by side-splash trays, or any combination thereof. Suitable fixed valve trays, sieve trays, dual flow trays, and grids can include those disclosed in Distillation Design, Henry Z. Kister, McGraw-Hill Inc., 1992, pages 262 to 265 and pages 464-466. Suitable jet tab trays can include those disclosed in WO Publication No. WO2011/014345. Suitable evaporation systems can be or can include one or more wiped film evaporators such as those described in Boung Wook Lee et al., Thin-Film Evaporator Model for Continuous Active Pharmaceutical Ingredient Manufacturing, Ind. Eng. Chem. Res. 2020, 57, 7, 3252-3260; Leonard E. Najder, Thin Film Evaporation, Ind. Eng. Chem. 1964, 56, 2, 26-30; and Jacinto Lopez-Toledo, Heat and Mass Transfer Characteristics of a Wiped Film Evaporator, Dissertation, The University of Texas at Austin, August 2006. Suitable crystallization systems can be or can include those described in H. J. M. Kramer, G. M. van Rosmalen, CRYSTALLIZATION, Encyclopedia of Separation Science, Academic Press, 2000, Pages 64-84.

At least a portion of the recycle stream in line 1042 can make up the recycle stream in line 1046. In some embodiments, a portion of the recycle stream in line 1042 can be purged via line 1048 from the system 100. In some embodiments, the recycle stream in line 1042 can be partially condensed or completely condensed and collected in a reflux drum or a drumless condenser and a portion of the collected condensed distillate can be refluxed back into the separation zone 1040. In some embodiments, when a portion of the recycle stream in line 1042 is purged via line 1048, the portion purged can be in the liquid phase and/or the vapor phase. At least a portion of the remainder of the collected condensed distillate can be recycled as the recycle stream via line 1046 to the reaction zone 1030. In some embodiments, the separation zone 1040, e.g., a distillation column, can be equipped with one or more reboilers that can operate at a temperature in a range of from 140° C., 145° C., 150° C., or 160° C. to 170° C., 180° C., or 190° C.

In some embodiments, the at least a portion of the overhead purged via line 1048 from the system 100 can include at least a portion of the one or more light compounds such as isoprene, piperylene, and/or benzene to avoid the build-up of such compounds within the system 100. For example, the recycle stream in line 1042 can be partially condensed if in the gaseous phase and/or partially vaporized if in the liquid phase and introduced into a separator to produce a gaseous overhead that can be separated via line 1048 and a liquid bottoms via line 1046 that can be recycled as the recycle stream to the reaction zone 1030. In some embodiments, other light compounds that can be purged via line 1048 from the system 100 can include molecular oxygen that can contaminate the hydrocarbon feed during storage and/or transport and/or can enter into the system 100 via the separation zone 1040, for example.

In some embodiments, the separation zone 1040, e.g., a distillation column, can operate under a vacuum to reduce or avoid fouling and/or to reduce or avoid cracking of tri-cyclopentadiene that may occur at a high bottom temperature. In some embodiments, the separation zone 1040, e.g., a distillation column, can be operated at a pressure ≤100 kPa-a, ≤80 kPa-a, ≤60 kPa-a, ≤40 kPa-a, ≤30 kPa-a, or ≤20 kPa-a. In other embodiments, however, the separation zone 1040, e.g., a distillation column, can be operated at ambient pressure.

In some embodiments, the first product stream in line 1044 can be cooled to a temperature in a range of from 30° C., 35° C., 40° C., or 45° C. to 50° C., 60° C., or 70° C. In some embodiments, the cooled first product stream in line 1044 can be sent to storage as a tri-cyclopentadiene or first product stream. In some embodiments, the first product stream in line 1044 can include residual di-cyclopentadiene, tri-cyclopentadiene, tetra-cyclopentadiene, optionally oligomers heavier than tetra-cyclopentadiene, or a mixture thereof. The first product stream in line 1044 can include 45 wt %, 50 wt %, 55 wt %, or 60 wt % to 70 wt %, 75 wt %, 80 wt %, or 85 wt % of tri-cyclopentadiene based on the total weight of the first product stream in line 1044. The first product stream in line 1044 can include 15 wt %, 20 wt %, 25 wt %, or 30 wt % to 40 wt %, 45 wt %, 50 wt %, or 55 wt % of residual di-cyclopentadiene based on the total weight of the first product stream in line 1044. The first product stream in line 1044 can include 0.1 wt %, 0.5 wt %, 1 wt %, 1.5 wt % or 2 wt % to 4 wt %, 5 wt %, 6 wt % or 7 wt % of tetra-cyclopentadiene and optionally one or more oligomers heavier than tetra-cyclopentadiene based on the total weight of the first product stream in line 1044. In some embodiments, the first product stream in line 1044 can include ≤7 wt %, ≤5 wt %, or ≤3 wt % of tetra-cyclopentadiene and optionally one or more oligomers heavier than tetra-cyclopentadiene based on the total weight of the first product stream in line 1044.

In some embodiments, a weight ratio of tri-cyclopentadiene to tetra-cyclopentadiene and optionally the one or more oligomers heavier than tetra-cyclopentadiene in the first product stream in line 1044 can be ≥5, ≥10, ≥15, ≥20, ≥25, ≥30, ≥35, ≥40, ≥45, ≥50, ≥55, or ≥60. In some embodiments, the first product stream in line 1044 can include ≥95 wt %, ≥96 wt %, ≥97 wt %, ≥98 wt %, or ≥99 wt % of a combined amount of tri-cyclopentadiene, residual di-cyclopentadiene, tetra-cyclopentadiene, and optionally one or more oligomers heavier than tetra-cyclopentadiene based on the total weight of the first product stream in line 1044. In some embodiments, a weight ratio of tri-cyclopentadiene to residual di-cyclopentadiene in the first product stream in line 1044 can be ≥1, ≥2, or ≥2.5.

FIG. 2 depicts an illustrative system 200 for making a composition containing tri-cyclopentadiene that includes the optional mixing zone 1020, the reaction zone 1030, and the separation zone 1040 shown in the system depicted in FIG. 1. The system 200 can further include one or more optional oxygen separation zones (two are shown, 2005, 2050), an optional pre-heat zone 2025, an optional dimerization zone 2070, a separation zone 2060, a purge feed separation zone 2075, one or more optional antioxidant feeds (five are shown, 2021, 2041, 2043, 2061, and 2065) and a diluent feed in line 2045, according to one or more embodiments.

As noted above, in some embodiments molecular oxygen can contaminate the hydrocarbon feed in line 1010 and it can be desirable to remove at least a portion of the molecular oxygen therefrom. In some embodiments, the hydrocarbon feed via line 1010 and a stripping gas deficient in molecular oxygen via line 2001 can be introduced into the oxygen separation zone 2005 and can counter currently flow through the oxygen separation zone 2005. The oxygen separation zone 2005 can include one or more internal components 2006 such as packing, trays, or a combination thereof, to promote mass transfer of the molecular oxygen from the hydrocarbon feed to the stripping gas. A molecular oxygen rich gas via line 2007 and a molecular oxygen lean hydrocarbon feed via line 2010 can be recovered from the oxygen separation zone 2005. In some embodiments, the amount of molecular oxygen removed from the hydrocarbon feed in line 1010 can be ≥90%, ≥93%, ≥95%, ≥97%, ≥98%, or ≥99% of any molecular oxygen present in the hydrocarbon feed in line 1010.

In other embodiments, the recycle stream in line 1042 recovered from the separation zone 1040 and a stripping gas deficient in molecular oxygen via line 2048 can be introduced into the oxygen separation zone 2050 and can counter currently flow through the oxygen separation zone 2050. In some embodiments, an optional antioxidant feed via line 2041 can be combined with the recycle stream in line 1042 to produce an antioxidant containing overhead via line 2042 that can be introduced into the oxygen separation zone 2050. It should be understood that the recycle stream in line 1042 can be in the liquid phase when introduced into the oxygen separation zone 2050. The recycle stream in line 1042 can be cooled within one or more heat exchange zones to condense the recycle stream. In some embodiments, the recycle stream in line 1042 can be an overhead vapor stream that can be partially condensed and separated into two or more streams. In some embodiments, the recycle stream in line 1042 can be an overhead vapor stream that can be partially condensed to provide a vapor stream that can be introduced to a vacuum system and removed from the system 200, a liquid stream that can be refluxed to the separation zone 1040, and a liquid stream that can be recycled via line 2046 to the reaction zone 1030.

The oxygen separation zone 2050 can include one or more internal components 2051 such as packing, trays, or a combination thereof, to promote mass transfer of the molecular oxygen from the overhead to the stripping gas. A molecular oxygen rich gas via line 2052 and a molecular oxygen lean product via line 2054 can be recovered from the oxygen separation zone 2050. In some embodiments, the amount of molecular oxygen removed from the recycle stream in line 1042 or 2042 can be ≥90%, ≥93%, ≥95%, ≥97%, ≥98%, or ≥99% of any molecular oxygen present in the recycle stream in line 1042. In some embodiments, the oxygen separation zone 2005 or the oxygen separation zone 2050 can be included in the system 200. In other embodiments, both the oxygen separation zone 2005 and the oxygen separation zone 2050 can be included in the system 200.

In some embodiments, the hydrocarbon feed via line 1010 or the molecular oxygen lean hydrocarbon feed via line 2010 and a recycle stream via line 2046 can be introduced into the optional mixing zone 1020 to produce a mixed feed via line 2023. The mixed feed via line 2023 can be introduced into the preheat zone 2025 to produce a preheated mixed feed via line 2027 that can be introduced into the reaction zone 1030. In some embodiments, the optional antioxidant feed via line 2021 can be combined with the mixed feed in in line 2023 to produce a mixed feed via line 2024 that includes the antioxidant. In such embodiment, the mixed feed in line 2024 can be introduced into the preheat zone 2025 to produce the preheated mixed feed via line 2027. In other embodiments, the optional antioxidant feed in line 2021 can be combed with the hydrocarbon feed in line 1010 and/or 2010 and/or the recycle stream in line 2046. The preheated mixed feed in line 2027 can be at a temperature in a range of from 145° C., 150° C., 155° C., or 160° C. to 165° C., 170° C., 175° C., or 180° C. when introduced into the reaction zone 1030.

The preheat zone 2025 can be operated at a sufficient pressure to maintain a liquid phase of the contents therein. In some embodiments, the preheat zone 2025 can be operated at a pressure of >138 kPa-absolute (kPa-a), >206 kPa-a, or >653 kPa-a and up to any pressure desired, e.g., up to a pressure of 1,540 kPa-a or 4,485 kPa-a, when operated at a temperature of 160° C. In some embodiments, the preheat zone 2025 can be operated at a >255 kPa-absolute (kPa-a), >351 kPa-a, or >653 kPa-a and up to any pressure desired, e.g., up to a pressure of 1,420 kPa-a or 4,400 kPa-a, when operated at a temperature of 190° C.

In some embodiments, some conversion of di-cyclopentadiene to tri-cyclopentadiene and higher oligomers can occur within the preheat zone 2025 but the temperature and residence time within the preheat zone can be controlled such that the amount of di-cyclopentadiene converted to tri-cyclopentadiene, tetra-cyclopentadiene, and optionally one or more oligomers heavier than tetra-cyclopentadiene, can be ≤1 wt %, ≤0.7 wt %, ≤0.5 wt %, or ≤0.3 wt %.

When the hydrocarbon feed in line 1010 or the molecular oxygen lean hydrocarbon feed in line 2010 and the recycle stream in line 2046 provide a mixed feed in line 2023 or upon separate introduction into the preheat zone 2025 provide a mixture that includes ≥97.5 wt %, ≥98 wt %, or ≥99 wt % of di-cyclopentadiene based on the combined amount of di-cyclopentadiene and any cyclopentadiene and the preheat zone 2025 is at a temperature of 175° C., an equilibrium amount of cyclopentadiene in a range of from 3 wt % to 4 wt %, e.g., 3.5 wt %, can be obtained. In some embodiments, the amount of cyclopentadiene may or may not reach equilibrium within the preheat zone 2025 but the amount of cyclopentadiene can be further increased within the reaction zone 1030.

The preheated mixed feed introduced via line 2027 into the reaction zone 1030 can be subjected to reaction conditions sufficient to effect reaction between the cyclopentadiene and the di-cyclopentadiene within the reaction zone 1030 to produce the effluent via line 1033 that includes residual cyclopentadiene, residual di-cyclopentadiene, tri-cyclopentadiene, tetra-cyclopentadiene, and optionally one or more oligomers heavier than tetra-cyclopentadiene, as described above with regard to FIG. 1. The effluent via line 1033 can be introduced into the separation zone 1040 to produce the overhead or recycle stream via line 1042 and the bottoms or first product stream via line 1044, as described above with regard to FIG. 1.

As shown in FIG. 2, in some embodiments, at least a portion of the first product stream in line 1044 can be sent to storage as a tri-cyclopentadiene product via line 2044. In some embodiments, if a portion of the first product stream in line 1044 is sent to storage via line 2044, one or more antioxidants via line 2043 can optionally be combined with the bottoms in line 2044.

In some embodiments, at least a portion of the first product stream in line 1044 can be introduced via line 2046 into the separation zone 2060. As shown, an overhead or second product via line 2062 and a bottoms or third product via line 2066 can be recovered from the separation zone 2060. In some embodiments, the overhead or second product in line 2062 can be partially condensed to produce a vapor stream that can be introduced into a vacuum system and removed from the system 200, a liquid stream that can be refluxed back into the separation zone 2060, and a liquid stream that can be recovered as the second product.

The separation zone 2060 can be or can include any suitable separation technique. In some embodiments, suitable separation techniques can be or can include, but are not limited to, distillation, evaporation, crystallization, or a combination thereof. In some embodiments, the separation zone 2060 can be or can include one or more distillation columns, one or more divided wall distillation columns, or a combination thereof. In some embodiments, the separation zone 2060 can include one or more internal structures to facilitate separation of the overhead and the bottoms from the bottoms in line 2046. The internal structure(s) can facilitate vapor/liquid separation and/or liquid collection. Illustrative internal structures can be or can include, but are not limited to, trays, grids, packing, or any combination thereof. Illustrative trays can include, but are not limited to, fixed valve trays, jet tab trays, sieve trays, dual flow trays, baffle trays, angle iron trays, draw off trays, shed deck trays, disk trays, donut trays, side by side-splash trays, or any combination thereof. Suitable fixed valve trays, sieve trays, dual flow trays, and grids can include those disclosed in Distillation Design, Henry Z. Kister, McGraw-Hill Inc., 1992, pages 262 to 265 and pages 464-466. Suitable jet tab trays can include those disclosed in WO Publication No. WO2011/014345. Suitable evaporation systems can be or can include those described in Boung Wook Lee et al., Thin-Film Evaporator Model for Continuous Active Pharmaceutical Ingredient Manufacturing, Ind. Eng. Chem. Res. 2020, 57, 7, 3252-3260; Leonard E. Najder, Thin Film Evaporation, Ind. Eng. Chem. 1964, 56, 2, 26-30; and Jacinto Lopez-Toledo, Heat and Mass Transfer Characteristics of a Wiped Film Evaporator, Dissertation, The University of Texas at Austin, August 2006. Suitable crystallization systems can be or can include those described in H. J. M. Kramer, G. M. van Rosmalen, CRYSTALLIZATION, Encyclopedia of Separation Science, Academic Press, 2000, Pages 64-84.

The overhead or second product in line 2062 can be rich in tri-cyclopentadiene and lean in tetra-cyclopentadiene and the one or more optional oligomers heavier than tetra-cyclopentadiene. The second product in line 2062 can also include di-cyclopentadiene. In some embodiments, the second product in line 2062 can include ≥95 wt %, ≥98 wt %, or ≥99 wt % of a combined amount of di-cyclopentadiene and tri-cyclopentadiene. In some embodiments, a weight ratio of tri-cyclopentadiene to di-cyclopentadiene in the second product can be ≥1, ≥2, or ≥2.5. The bottoms or third product in line 2066 can be rich in tetra-cyclopentadiene and the one or more optional oligomers heavier than tetra-cyclopentadiene.

In some embodiments, the optional diluent feed via line 2045 can be combined with the at least a portion of the bottoms in line 2046 to produce a diluent rich bottoms via line 2047 that can be introduced into the separation zone 2060. The diluent in line 2045 can reduce or suppress a melting point of tetra-cyclopentadiene and reduce or suppress a melting point of the one or more oligomers heavier than tetra-cyclopentadiene within the separation zone 2060. In some embodiments, the diluent in line 2045 can be or can include, but is not limited to, one or more C₉₊ paraffins, C₉₊ iso-paraffins, C₉₊ aromatics, C₉₊ naphthenes, C₉₊ alpha-olefins, or a mixture thereof.

In some embodiments, the optional antioxidant feed via line 2061 can be combined with the second product in line 2062 to produce a second product via line 2063 that includes the antioxidant feed. In some embodiments, the optional antioxidant feed via line 2065 can be combined with the third product in line 2066 to produce a third product via line 2068 that includes the antioxidant feed. The optional antioxidant feeds that can be introduced via lines 2021, 2041, 2043, 2061, and/or 2065 can be or can include, but are not limited to, butylated hydroxytoluene, triphenyl phosphine, 4-tert-butylcatechol, phenylenediamine, or a mixture thereof.

Returning to the recycle stream in line 1042 and/or 2042, the recycle stream via line 1042 and/or 2042 can be introduced directly into the optional dimerization zone 2070. In other embodiments, the molecular oxygen lean product via line 2054 can be introduced into the optional dimerization zone 2070. In some embodiments, the dimerization zone 2070 can be operated at a temperature in a range of from 70° C., 75° C., 80° C., 85° C., 90° C., 100° C., or 105° C. to 110° C., 120° C., 125° C., 130° C., or 135° C. The dimerization zone 2070 can be operated at a pressure sufficient to maintain the contents therein in a liquid phase. In some embodiments, the dimerization zone 2070 can be operated at a temperature of ≥15 kPa-a, ≥44 kPa-a, or ≥790 kPa-a and up to any pressure desired, e.g., up to a pressure of 1,800 kPa-a or 4,730 kPa-a, when operated at a temperature of 90° C. In other embodiments, the dimerization zone 2070 can be operated at a temperature of ≥40 kPa-a, ≥113 kPa-a, or ≥790 kPa-a and up to any pressure desired, e.g., up to a pressure of 1,650 kPa-a or 4,570 kPa-a, when operated at a temperature of 130° C. In some embodiments, the residence time of the feed introduced via line 1042, 2042, and/or 2054 into the dimerization zone 2070 can be in a range of from 10 minutes, 20 minutes, 30 minutes, 45 minutes, 60 minutes, or 70 minutes to 100 minutes, 150 minutes, 200 minutes, 250 minutes, or 300 minutes.

In some embodiments, the recycle stream in line 1042 and/or 2042 and/or the molecular oxygen lean product via line 2054 can include cyclopentadiene in an amount of from 1 wt %, 3 wt %, 5 wt %, 10 wt %, 15 wt %, 20 wt %, 25 wt %, 30 wt %, 35 wt %, or 40 wt % to 50 wt %, 60 wt %, 65 wt %, 70 wt %, 75 wt %, 80 wt %, 85 wt %, 90 wt %, or 95 wt %, based on the total weight of the feed in line 1042, 2042, and/or 2054. Within the dimerization zone 2070 at least a portion of the residual cyclopentadiene therein can dimerize to produce a dimerized product via line 2072 that can include less cyclopentadiene than the feed introduced via line 1042, 2042, and/or 2054 into the dimerization zone 2070. In some embodiments, the dimerized product in line 2070 can include cyclopentadiene in a range of from 1 wt %, 2 wt %, or 3 wt %, to 3.5 wt %, 4 wt %, or 5 wt %, based on the total weight of the dimerized product in line 2070. In some embodiments, the residence time and temperature can be controlled to produce the dimerized product in line 2070 that includes a desired concentration of cyclopentadiene and di-cyclopentadiene with respect to one another. In some embodiments, when the feed introduced via line 1042, 2042, or 2054 into the dimerization zone 2070 includes 6.5 wt % of residual cyclopentadiene and the dimerization zone 2070 is operated at a temperature in a range of from 75° C. to 85° C., 85° C. to 95° C., or 115° C. to 125° C., the residence time within the dimerization zone 2070 can be in a range of from 45 minutes to 65 minutes, 20 minutes to 35 minutes, or 3 minutes to 10 minutes, respectively, to produce a dimerized product in line 2070 that can include about 2 wt % to about 5 wt % or about 3 wt % to about 4 wt % of cyclopentadiene. In other embodiments, when the feed introduced via line 1042, 2042, or 2054 into the dimerization zone 2070 includes >90 wt % of residual cyclopentadiene and the dimerization zone 2070 is operated at a temperature in a range of from 75° C. to 85° C., 85° C. to 95° C., or 115° C. to 125° C., the residence time within the dimerization zone 2070 can be in a range of from 215 minutes to 255 minutes, 115 minutes to 145 minutes, or 15 minutes to 40 minutes, respectively, to produce a dimerized product in line 2070 that can include about 2 wt % to about 5 wt % or about 3 wt % to about 4 wt % of cyclopentadiene.

In some embodiments, the dimerized product via line 2072 can be introduced into the optional separation zone 2075 to separate at least a portion of the light compounds, e.g., isoprene, piperylene, and/or benzene, from the system 200. A light compound containing purge steam via line 2048 can be recovered from the separation zone 2075 and removed from the system. A recycle stream via line 2046 that includes the cyclopentadiene and di-cyclopentadiene can be recovered from the separation zone 2075 and recycled to the optional mixing zone 1020, the optional preheat zone 2025, and/or the reaction zone 1030. In other embodiments, it should be understood that the optional separation zone 2075 can be located upstream of the dimerization zone 2070 such that at least a portion of the light compounds can be purged from the system 200 prior the dimerization zone 2070.

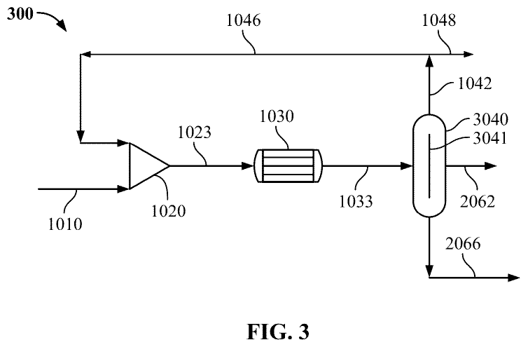

FIG. 3 depicts an illustrative system 300 for making a composition containing tri-cyclopentadiene that includes the optional mixing zone 1020, the reaction zone 1030, and a separation zone 3040, according to one or more embodiments. In some embodiments, the hydrocarbon feed via line 1010 and the recycle stream via line 1046 can be introduced into the mixing zone 1020 to produce the mixed feed via line 1023. The mixed feed via line 1023 can be introduced into the reaction zone 1030 to produce the effluent via line 1033, as described above with reference to FIG. 1. In other embodiments, the hydrocarbon feed via line 1010 and the recycle stream via line 1046 can be separately introduced into the reaction zone 1030 to produce the effluent via line 1033, as described above with reference to FIG. 1.

The effluent via line 1033 can be introduced into the separation zone 3040. As shown, the separation zone 3040 includes a distillation column that includes a dividing wall 3041 therein. As such, the separation zone 3040 can separate the effluent into the overhead or recycle stream via line 1042, the second product via line 2062, and the third product via line 2066, which can each have substantially the same compositions as described above with reference to FIG. 2. The dividing wall 3041 can provide multiple zones within the separation zone 3040 that can be operated at different temperatures to produce the recycle stream line 1042, the second product via line 2062 as a side draw, and the third product via line 2066 as a bottoms. In some embodiments, an optional diluent can be combined with the effluent in line 1033 and/or introduced separately into the separation zone 3040. The diluent, if used, can reduce or suppress a melting point of tetra-cyclopentadiene and reduce or suppress a melting point of the one or more oligomers heavier than tetra-cyclopentadiene within the separation zone 3040. In some embodiments, the diluent can be or can include, but is not limited to, one or more C₉₊ paraffins, C₉₊ iso-paraffins, C₉₊ aromatics, C₉₊ naphthenes, C₉₊ alpha-olefins, or a mixture thereof. In some embodiments, suitable separation zones 3040 that include a dividing wall 3041 can include those disclosed in I. J. Halvorsen, I. Dejanović, S. Skogestad, Ž. Olujić, Internal Configurations for a Multi-product Dividing Wall Column, Chemical Engineering Research and Design, Volume 91, Issue 10, 2013, Pages 1954-1965; I. J. Halvorsen, S. Skogestad, I. Dejanović, L. Matijašević, Ž. Olujić, Multi-Product Dividing Wall Columns: A Simple and Effective Assessment and Conceptual Design Procedure; S. Tututi-Avila, L. A. Domínguez-Díaz, N. Medina-Herrera, A. Jiménez-Gutiérrez, J. Hahn, Dividing-wall Columns: Design and Control of a Kaibel and a Satellite Distillation Column for BTX Separation, Chemical Engineering and Processing: Process Intensification, Volume 114, 2017, Pages 1-15; Ž. Olujić, M. Jödecke, A. Shilkin, G. Schuch, B. Kaibel, Equipment Improvement Trends in Distillation, Chemical Engineering and Processing: Process Intensification, Volume 48, Issue 6, 2009, Pages 1089-1104; and I. Dejanović, Lj. Matijašević, Ž. Olujić, Dividing Wall Column—A Breakthrough Towards Sustainable Distilling, Chemical Engineering and Processing: Process Intensification, Volume 49, Issue 6, 2010, Pages 559-580; and U.S. Pat. Nos. 1,915,681; 2,134,882; 2,295,256; 2,471,134; 3,058,893; 3,314,879; 3,412,016; 4,230,533; 4,994,152; 5,339,648; 5,580,425; 5,585,046; 5,709,780; 5,755,993; 5,785,819; 5,836,174; 5,897,748; 5,902,460; 5,914,012; 5,946,942; 6,077,985; 6,166,279; 6,250,106; 6,347,533; 6,395,950; 6,395,951; 6,407,303; 6,479,720; 6,483,002; 6,540,907; 6,551,465; 6,558,515; 6,645,350; 6,726,835; 6,770,173; 6,628,468; 6,846,389; 6,884,324; 6,927,314; 6,930,206; 6,958,111; 7,001,490; 7,005,057; 7,090,748; 7,108,770; 7,112,707; 7,118,653; 7,132,038; 7,169,267; 7,234,691; 7,264,696; 7,267,746; 7,287,747; 7,342,134; 7,357,378; 7,527,712; 7,528,290; 7,547,378; 7,556,717; U.S. Patent Application Publication Nos.: 2001/0010286; 2001/0052453; 2003/0230476; 2004/0204614; 2006/0005574; 2006/0137967; 2007/0293688; 2008/0081937; 2008/0289946; and 2009/0139852; EP Patent Application Publication Nos.: EP0122367A2; EP0126288A2; EP0780147A2; EP0806406A1; German Patent Application Publication and Patent Nos.: DE3522234C3; DE4336983A1; DE3510365C2; DE4336984C2; DE4336986C2; DE10135585C1; DE102004024688(A1); and WO Publication Nos.: WO 2003/051799; WO 2004/071618; WO 2005/046831; and WO 2009/092682.

It should be understood that the system 300 can also include one or more of the optional oxygen separation devices 2005, 2050, the optional preheat zone 2025, the optional dimerization reactor 2070, the optional separation zone 2075, one or more of the optional antioxidant feeds in lines 2021, 2041, 2043, 2061, and 2065, and/or the optional diluent feed in line 2045 described above with reference to FIG. 2.

### Listing of Embodiments

This disclosure may further include the following non-limiting embodiments.

A1. A process for making tri-cyclopentadiene, comprising: (I) feeding a hydrocarbon feed comprising cyclopentadiene and di-cyclopentadiene into a reaction zone; (II) subjecting the hydrocarbon feed to reaction conditions sufficient to effect reaction between the cyclopentadiene and the di-cyclopentadiene within the reaction zone to produce an effluent comprising residual cyclopentadiene, residual di-cyclopentadiene, tri-cyclopentadiene, and tetra-cyclopentadiene; (III) separating from the effluent a recycle stream comprising residual cyclopentadiene and a first portion of the residual di-cyclopentadiene, a second product comprising a second portion of the residual di-cyclopentadiene that is rich in tri-cyclopentadiene and lean in tetra-cyclopentadiene, and a third product that is rich in tetra-cyclopentadiene and lean in tri-cyclopentadiene; and (IV) feeding at least a portion of the recycle stream into the reaction zone.

A2. The process of A1, wherein the effluent comprises: 0.1 wt % to 6 wt % of TCPD-7; 5 wt % to 25 wt % of TCPD-3; 15 wt % to 30 wt % of TCPD-5; and 55 wt % to 75 wt % of TCPD-1, wherein all wt % values are based on the combined weight of TCPD-7, TCPD-3, TCPD-5, and TCPD-1 in the effluent.

A3. The process of A1 or A2, wherein the hydrocarbon feed and the recycle stream fed into the reaction zone comprise 0.1 wt %, 1 wt %, 3 wt %, or 5 wt % to 10 wt %, 30 wt %, or 50 wt % of the cyclopentadiene and 50 wt %, 70 wt %, or 90 wt % to 95 wt %, 97 wt %, 99 wt %, or 99.9 wt % of the di-cyclopentadiene based on the combined amount of the cyclopentadiene and the di-cyclopentadiene in the hydrocarbon feed and the recycle stream.

A4. The process of any of A1 to A3, wherein the reaction zone is adiabatic, isothermal, or a combination of adiabatic and isothermal.

A5. The process of any one of A1 to A4, further comprising: (V) pre-heating the hydrocarbon feed and the recycle stream either separately or as a mixture thereof to a temperature in a range from 155° C., 160° C., or 165° C. to 170° C., 175° C., or 180° C. before the hydrocarbon feed, the recycle stream, or the mixture thereof is fed into the reaction zone in step (I).

A6. The process of any one of A1 to A5, wherein the reaction conditions comprise a temperature in a range from 160° C., 165° C., or 170° C. to 180° C., 185° C., or 190° C. and a pressure sufficient to maintain the compounds within the reaction zone in a liquid phase.

A7. The process of any one of A1 to A6, wherein <15 wt %, preferably <10 wt %, or more preferably <7.5 wt % of the di-cyclopentadiene in the hydrocarbon feed is converted to tri-cyclopentadiene or to tetra-cyclopentadiene.

A8. The process of any one of A1 to A7, further comprising: (VI) feeding at least a portion of the recycle stream into a dimerization zone to effect conversion of at least a portion of the cyclopentadiene to di-cyclopentadiene to produce a dimerized recycle stream that is fed to the reaction zone in step (IV).

A9. The process of A8, wherein the dimerized recycle stream comprises 1 wt %, 2.5 wt %, or 3 wt % to 4 wt %, 5 wt %, or 6 wt % of cyclopentadiene based on the weight of the dimerized recycle stream.

A10. The process of any one of A1 to A9, wherein the hydrocarbon feed, the recycle stream, or both the hydrocarbon feed and the recycle stream comprise molecular oxygen, the process further comprising: (VII) stripping at least a portion of any oxygen present in the hydrocarbon feed to produce an oxygen-lean hydrocarbon feed that is fed into the reaction zone; (VIII) stripping at least a portion of any oxygen present in the recycle stream to produce an oxygen-lean recycle stream that is fed into the reaction zone; or (IX) carrying out both steps (VII) and (VIII), wherein the at least a portion of any oxygen present in the hydrocarbon feed, the recycle stream, or both the hydrocarbon feed and the recycle stream is stripped with a stripping gas comprising nitrogen, one or more C₁-C₅ hydrocarbons, or a mixture thereof.

A11. The process of any one of A1 to A10, wherein the second product comprises ≥95 wt %, preferably ≥98 wt %, or more preferably ≥99 wt % of a combined amount of di-cyclopentadiene and tri-cyclopentadiene.

A12. The process of any one of A1 to A11, wherein a weight ratio of tri-cyclopentadiene to di-cyclopentadiene in the second product is ≥1, preferably ≥2, or more preferably ≥2.5.

A13. The process of any one of A1 to A11, wherein di-cyclopentadiene in the hydrocarbon feed and the recycle stream fed into the reaction zone, combined, include a mixture of endo-di-cyclopentadiene and exo-di-cyclopentadiene.

A14. The process of A13, wherein a weight ratio of the endo-di-cyclopentadiene to the exo-di-cyclopentadiene fed into the reaction zone is in a range of from 4.5, 5, 5.5, 6, or 7 to 8, 9, 9.5, 10, 12, 14, 16, 18, or 20.

Various terms have been defined above. To the extent a term used in a claim is not defined above, it should be given the broadest definition persons in the pertinent art have given that term as reflected in at least one printed publication or issued patent. Furthermore, all patents, test procedures, and other documents cited in this application are fully incorporated by reference to the extent such disclosure is not inconsistent with this application and for all jurisdictions in which such incorporation is permitted.

While the foregoing is directed to embodiments of the present invention, other and further embodiments of the invention may be devised without departing from the basic scope thereof, and the scope thereof is determined by the claims that follow.

1. A composition, comprising:
0.1 wt % to 6 wt % of TCPD-7 having a structure of 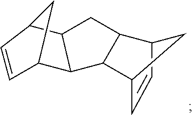 [SMILES: C1=CC2CC1C1CC3C4C=CC(C4)C3C21]
5 wt % to 25 wt % of TCPD-3 having a structure of 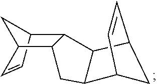 [SMILES: C1=CC2CC1C1CC3C4C=CC(C4)C3C21]
15 wt % to 30 wt % of TCPD-5 having a structure of 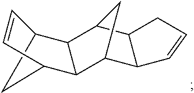 [SMILES: C1=CC2C(C1)C1CC2C2C3C=CC(CC3)C12]
and
55 wt % to 75 wt % of TCPD-1 having a structure of 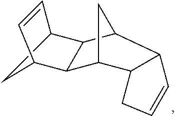 [SMILES: C1=CC2C(C1)C1CC2C2C3C=CC(CC3)C12]
wherein all wt % values are based on the combined weight of TCPD-7, TCPD-3, TCPD-5, and TCPD-1 in the composition.

2. The composition of claim 1, further comprising di-cyclopentadiene.

3. The composition of claim 2, wherein the composition comprises ≥97 wt % of a combined amount of di-cyclopentadiene and tri-cyclopentadiene, based on the total weight of the composition.

4. The composition of claim 2, wherein a weight ratio of tri-cyclopentadiene to di-cyclopentadiene is ≥1.

5. The composition of claim 1, further comprising di-cyclopentadiene and tetra-cyclopentadiene.

6. The composition of claim 5, wherein the composition comprises 50 wt % to 80 wt % of tri-cyclopentadiene, 20 wt % to 50 wt % of di-cyclopentadiene, and 0.1 wt % to 7 wt % of tetra-cyclopentadiene, based on the total weight of the composition.

7. The composition of claim 5, wherein a weight ratio of tri-cyclopentadiene to tetra-cyclopentadiene is >10.

8. The composition of claim 5, wherein the composition comprises ≥97 wt % of a combined amount of tri-cyclopentadiene, di-cyclopentadiene, and tetra-cyclopentadiene, based on the total weight of the composition.

9. The composition of claim 5, wherein the composition comprises ≤7 wt % of tetra-cyclopentadiene, based on the total weight of the composition.

10. The composition of claim 5, wherein a weight ratio of tri-cyclopentadiene to di-cyclopentadiene in the composition is ≥1.

11. A process for making tri-cyclopentadiene, comprising:
(I) feeding a hydrocarbon feed comprising cyclopentadiene and di-cyclopentadiene into a reaction zone;
(II) subjecting the hydrocarbon feed to reaction conditions sufficient to effect reaction between the cyclopentadiene and the di-cyclopentadiene within the reaction zone to produce an effluent comprising residual cyclopentadiene, residual di-cyclopentadiene, tri-cyclopentadiene, and tetra-cyclopentadiene;
(III) separating from the effluent a recycle stream comprising residual cyclopentadiene and residual di-cyclopentadiene and a first product stream comprising residual di-cyclopentadiene, tri-cyclopentadiene, and tetra-cyclopentadiene; and
(IV) feeding at least a portion of the recycle stream into the reaction zone.

12. The process of claim 11, wherein the tri-cyclopentadiene in the effluent comprises:
0.1 wt % to 6 wt % of TCPD-7 having a structure of 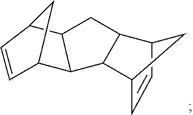 [SMILES: C1=CC2CC1C1CC3C4C=CC(C4)C3C21]
5 wt % to 25 wt % of TCPD-3 having a structure of 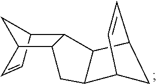 [SMILES: C1=CC2CC1C1CC3C4C=CC(C4)C3C21]
15 wt % to 30 wt % of TCPD-5 having a structure of 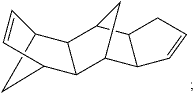 [SMILES: C1=CC2C(C1)C1CC2C2C3C=CC(CC3)C12]
and
55 wt % to 75 wt % of TCPD-1 having a structure of 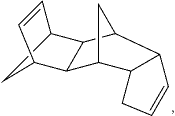 [SMILES: C1=CC2C(C1)C1CC2C2C3C=CC(CC3)C12]
wherein all wt % values are based on the combined weight of TCPD-7, TCPD-3, TCPD-5, and TCPD-1 in the effluent.

13. The process of claim 12, wherein TCPD-7, TCPD-3, TCPD-5, and TCPD-1, in total, constitute at least 95 wt % of all tri-cyclopentadiene in the effluent.

14. The process of claim 11, wherein the first product stream comprises 50 wt % to 80 wt % of tri-cyclopentadiene, 20 wt % to 50 wt % of di-cyclopentadiene, and 0.1 wt % to 7 wt % of tetra-cyclopentadiene, based on the total weight of the first product stream.

15. The process of claim 11, wherein the hydrocarbon feed and the recycle stream fed into the reaction zone comprise 0.1 wt % to 50 wt % of cyclopentadiene and 50 wt % to 99.9 wt % of di-cyclopentadiene, based on the total combined amount of the cyclopentadiene and the di-cyclopentadiene in the hydrocarbon feed and the recycle stream.

16. The process of claim 15, wherein a molar ratio of di-cyclopentadiene to cyclopentadiene, based on the total combined amount of the cyclopentadiene and the di-cyclopentadiene in the hydrocarbon feed and the recycle stream, is in a range of from 4.5 to 49.5.

17. The process of claim 11, wherein a residence time within the reaction zone is in a range of from 10 minutes to 70 minutes, and wherein the reaction zone is adiabatic, isothermal, or a combination of adiabatic and isothermal.

18. The process of claim 11, wherein di-cyclopentadiene in the hydrocarbon feed and the recycle stream fed into the reaction zone, combined, comprise a mixture of endo-di-cyclopentadiene and exo-di-cyclopentadiene, and wherein a weight ratio of endo-di-cyclopentadiene to exo-di-cyclopentadiene fed into the reaction zone is in a range of from 4.5 to 20.

19. The process of claim 11, further comprising:
(V) pre-heating the hydrocarbon feed and the recycle stream either separately or as a mixture thereof to a temperature in a range from 155° C. to 180° C. before the hydrocarbon feed, the recycle stream, or the mixture thereof is fed into the reaction zone in step (I), wherein:
the reaction conditions comprise a temperature in a range from 160° C. to 190° C. and a pressure sufficient to maintain the compounds within the reaction zone in a liquid phase, and
<15 wt % of the di-cyclopentadiene in the hydrocarbon feed is converted to tri-cyclopentadiene and/or tetra-cyclopentadiene.

20. The process of claim 11, further comprising:
(VI) feeding at least a portion of the recycle stream into a dimerization zone to effect conversion of at least a portion of the residual cyclopentadiene to di-cyclopentadiene to produce a dimerized recycle stream that is fed to the reaction zone in step (IV).

21. The process of claim 20, wherein the dimerized recycle stream comprises 1 wt % to 6 wt % of residual cyclopentadiene, based on the weight of the dimerized recycle stream.

22. The process of claim 11, wherein the hydrocarbon feed, the recycle stream, or both the hydrocarbon feed and the recycle stream comprise molecular oxygen, the process further comprising:
(VII) stripping at least a portion of any oxygen present in the hydrocarbon feed to produce an oxygen-lean hydrocarbon feed that is fed into the reaction zone;
(VIII) stripping at least a portion of any oxygen present in the recycle stream to produce an oxygen-lean recycle stream that is fed into the reaction zone; or
(IX) carrying out both steps (VII) and (VIII), wherein the at least a portion of any oxygen present in the hydrocarbon feed, the recycle stream, or both the hydrocarbon feed and the recycle stream is stripped with a stripping gas comprising nitrogen, one or more C₁-C₅ hydrocarbons, or a mixture thereof.

23. The process of claim 11, further comprising:
(X) separating from the first product stream a second product that is rich in tri-cyclopentadiene and lean in tetra-cyclopentadiene and a third product that is rich in tetra-cyclopentadiene and lean in tri-cyclopentadiene.

24. The process of claim 23, wherein the second product further comprises residual di-cyclopentadiene, and wherein the second product comprises ≥95 wt % of a combined amount of residual di-cyclopentadiene and tri-cyclopentadiene.

25. The process of claim 24, wherein a weight ratio of tri-cyclopentadiene to residual di-cyclopentadiene in the second product is ≥1.

26. The process of claim 23, wherein the second product and the third product are separated from the first product stream by distillation, evaporation, crystallization, or a combination thereof.

27. The process of claim 26, wherein:
the second product and the third product are separated from the first product stream by distillation within a distillation column,
a diluent is introduced into the distillation column to suppress a melting point of the tetra-cyclopentadiene, and
the diluent comprises one or more C₉₊ paraffins, C₉₊ iso-paraffins, C₉₊ aromatics, C₉₊ naphthenes, C₉₊ alpha-olefins, or a mixture thereof.

28. The process of claim 23, further comprising:
(XI) combining an anti-oxidant with at least one of: the hydrocarbon feed, the recycle stream, the first product stream, the second product, and the third product, wherein the anti-oxidant comprises butylated hydroxytoluene, triphenyl phosphine, 4-tert-butylcatechol, phenylenediamine, or a mixture thereof.
